(19) United States
(12) Patent Application Publication
(10) Pub. No.: US 2022/0062458 A9
(43) Pub. Date: Mar. 3, 2022

(54) Saturated steam sterilization device and process having improved sterilization reliability and temperature control

(71) Applicant: Milton E. Pedrazzi, Pleasanton, CA (US)

(72) Inventor: Milton E. Pedrazzi, Pleasanton, CA (US)

(21) Appl. No.: 15/998,047

(22) Filed: Jun. 18, 2018

Related U.S. Application Data
(63) Continuation of application No. 14/392,015, filed on Jan. 27, 2015, now abandoned.
(60) Provisional application No. 61/741,923, filed on Jul. 30, 2012.

(51) Int. Cl. A61L 2/07; A61L 2/26

(52) U.S. Cl. CPC A61L 2/07; A61L 2/26; A61L 2202/24; A61L 2202/14; A61L 2202/122

(57) ABSTRACT

Sterilization devices and procedures are described having improved temperature sensing and feedback control systems such that temperature control of about ±1 deg. C is achieved. Independent pressure measurement ensures that saturated steam has been achieved before the start of the sterilization procedure and is maintained throughout. More reliable sterilization is achieved thereby without risking damage to temperature-sensitive components of the instrument being sterilized.

### CROSS-REFERENCE TO RELATED APPLICATIONS

This application claims priority from US provisional patent application Ser. No. 61/741,923 filed 30 Jul. [[30]] 2012, international application number PCT/US2013/000176 having international filing date 29 Jul. 2013, and U.S. patent application Ser. No. 14/392,015 having filing date 27 Jan. 2015, pursuant to one or more of 35 U.S.C. § 119, 120, 365 or other provisions of the United States Code. The entire contents of the cited applications are included herein by reference for all purposes.

### BACKGROUND OF THE INVENTION

### 1. Field of Invention

This invention relates generally to the field of methods and devices for sterilization, more particularly to methods and devices for steam sterilization of medical, dental and/or veterinarian devices, and most particularly to saturated steam sterilization of dental handpieces having improved sterilization reliability and temperature control.

### 2. Description of Prior Art

It is a clear imperative in modern medical, dental and veterinarian practice that all instruments be sterile prior to use whenever such instruments have at least some chance to penetrate the skin of the patient (including non-human patients) or otherwise to come into contact with internal bodily fluids of the patient. It is also good practice in many cases to sterilize instruments following use to help insure safe disposal even if such instruments are not intended for re-use.

The same prudent sterilization procedures should also be employed in fields such as manicure, pedicure, tattooing, acupuncture, biopsy, among other procedures in which contact with patient's body fluids may occur.

Typical examples of instruments calling for one or more sterilizations over their useful service life include dental handpieces, scalpels and the full warehouse of surgical instruments, endoscopes, proctoscopes, laparoscopes, biopsy probes, acupuncture needles, instruments used by manicurists and pedicurists, tattoo artist's needles, veterinary instruments, among many others.

Failure to use sterilized instruments may expose the patient to a risk of infection, which may impede their recovery, produce serious additional medical difficulties or, at worst, cause death. In order to avoid the risk of infection, it is therefore of the utmost importance that all those processes which are designed to produce sterile instruments be carried out efficiently, reproducibly and reliably. It is one object of the present invention to describe devices and procedures for more reliable, efficient and effective sterilization.

To be concrete in our descriptions, we direct primary attention to the sterilization of dental handpieces. However, this is by way of illustration and not limitation since a person having ordinary skill in the art will readily appreciate how the devices and procedures described herein are readily applicable to other sterilizations.

Although we are aware of no precise epidemiological studies, it is generally accepted in the field of dentistry that dental handpieces have the potential to transmit disease from patient-to-patient or from patient-to-dental office personnel. During its use, the various dental handpiece regions and components, including the turbine, and external surfaces, can become repositories for blood, oral debris, soft tissue and microbes. Some of these microbes can be pathologic. Current methods of sterilization and cleaning may not always and reliably produce a clean and sterile handpiece for use on the next patient. The improved sterilization devices and procedures described herein provide an improved ability to clean and reliably sterilize the inside and outside of the handpiece during the sterilization procedure. Cleaning and sterilizing handpieces during the sterilization procedure thus reliably provides a clean and a sterile handpiece for each patient.

Current heat sterilization techniques and devices typically suffer from disadvantages such as inadequate control of the heating process, inconvenient turn-around times, dangers to the operators of the device, among other disadvantages. Reducing or eliminating at least one of these disadvantages is among the objectives of the present invention. Typical examples of prior art sterilization devices include U.S. Pat. Nos. 4,376,096 (“'096”), 7,018,592 (“'592”) and 5,520,892 (“'892”).

Both overheating and underheating are undesirable in a sterilization process. Overheating tends to damage the dental handpiece (or temperature-sensitive components of other devices undergoing sterilization), and in particular tends to damage the turbine assembly located in the head of the handpiece. This is primarily due to the inability of typical present day sterilizers to control heat precisely, tending to overheat the handpieces during the sterilization cycle. The ability of the sterilization unit to control temperature accurately throughout the sterilization cycle is thus an important method to reduce the possibility of damage to the handpiece and to its turbine assembly. Improved temperature control is among the objectives of the present invention.

Thus, it should be emphasized that numerous sterilization procedures are adequate to sterilize devices having no components sensitive to very high temperatures, such as (stainless steel) surgical scalpels and the like. Steam temperatures greatly exceeding that needed for sterilization can be employed to ensure complete sterilization, although with less longer turnaround time for the instruments to cool from a higher temperature. Controlling the sterilization process becomes more challenging when there is an upper limit to the temperature that can be used, as with typical dental handpieces. The processes described herein also have advantages in reducing the turnaround time before instruments can be safely reused, and avoiding the use of pressures in excess of that required for producing and maintaining saturated steam at the desired temperature

Saturated steam provides perhaps the most direct sterilization method for handpieces and other metal or comparable instruments. Saturated steam sterilization is recommended by the Food and Drug Administration (FDA), the Center for Disease Control (CDC), and the American Dental Association (ADA) because of its ability to reliably kill microbes when used for the proper amount of time under adequate conditions of pressure, humidity and temperature. Other conventional steam/pressure/heat sterilizers, including but not limited to autoclaves, typically generate temperatures that are sufficiently elevated to damage the turbine assembly of dental handpieces. Accordingly, this damage to the turbine assembly of dental handpieces results in a higher cost to dentists, patients and insurers because of the necessity of repairing or replacing damaged handpieces, hence a higher cost of treatment.

It is commonly accepted that in order to assure that a device has achieved saturated steam; the device must produce steam temperatures and pressures in agreement with the steam tables of ASME (American Society of Mechanical Engineers). In addition, a commercial sterilizer intended for the US market should satisfy the FDA's criteria for saturated steam, which largely derive from the AAMI (Association for the Advancement of Medical Instrumentation). These organizations point to a definition that states, in essence, if steam has a pressure and temperature in agreement with the ASME steam tables, it is correct to claim that saturated steam has been produced.

Current practice calls for dental handpieces to be sterilized anew for use on each patient. Thus, another substantial disadvantage of many conventional sterilizers arises, for example, their relatively long turn-around-time. Some sterilizers take as long as '40 minutes or longer to accomplish the sterilization cycle. Other disadvantages arise for those sterilizers that require the use of bags to sterilize handpieces. In addition to bags being a deterrent to sterilization and storage, the bags prevent or impede the ability to flush the dental handpieces with saturated steam. Flushing the handpieces with saturated steam enables the internal aspects of the handpieces to be more thoroughly sterilized and cleaned.

Current sterilizers often overheat the dental handpieces which can easily damage internal (often movable) components, driving up costs to the dental or medical professional, their employer and ultimately their patients or insurers. Unless a steam sterilizer can reliably reproduce the same physical conditions in every sterilization cycle it cannot qualify as an effective and safe saturated steam sterilizer. The production of saturated steam at a substantially constant temperature and pressure during the entire sterilization process provides the ability to reliably reproduce the physical conditions within the sterilization chamber. However, since temperature and pressure are dependently related for saturated steam, they cannot be selected independently while maintaining a saturated steam vapor.

Thus, there is a need in the art for sterilization devices and procedures for medical, dental and similar instruments that control the heating sufficiently accurately to cause reliable, reproducible sterilization of the instruments without excessive overheating and the corresponding risk of damage to the instruments, and advantageously have reasonably short turnaround times.

### SUMMARY OF THE INVENTION

Accordingly and advantageously, the present invention relates to devices and methods for sterilization of medical, dental and similar instruments in a reliable and reproducible manner under accurate control of heating and cooling processes, including adequate temperature control to avoid damage by overheating to heat-sensitive components of the item to be sterilized.

Accordingly and advantageously, it is an object of the present invention to provide heat sterilization devices and/or methods using saturated steam as would be advantageous for the sterilization of dental handpieces and other instruments or tools. Such devices and/or methods as described herein have sufficiently accurate and reliable heat control such that reliable and reproducible sterilization occurs on all parts of the instrument or tool, no damage to the instrument occurs, and which requires a relatively short total sterilization cycle time including cooling to a reusable or patient-ready condition, typically not exceeding approximately 23 minutes.

It is another object of the present invention to provide a heat sterilization unit using saturated steam under steady state conditions recognizing thereby that steady state conditions are one advantageous way to ensure that the desired physical conditions are achieved throughout the sterilizer.

It is yet another object of the present invention to improve the reliability of the sterilization process by, among other techniques, monitoring both temperature and pressure. It is shown that separate determination of temperature or pressure can lead to deceptive indications of proper sterilization when, in fact, sterilization may not have occurred.

Some embodiments include a novel heat control system that produces temperatures in the sterilization chamber so as to create saturated steam, and its resultant pressure, and to do so in a time frame that sterilizes typical dental handpieces without damaging the turbine assembly. This heat control system enables accurate temperature control at substantially any desired temperature for substantially any length of time. Accurate heat control is necessary to produce saturated steam reliably and reproducibly, and saturated steam is the most commonly employed method of sterilizing instruments. In some embodiments, mineral-free water in the form of liquid water, steam and saturated steam is flushed through the handpiece, tubing and turbine assembly during the sterilization cycle, removing debris and ensuring the thorough sterilization of the handpieces inside and out. The sterilization system in some particular embodiments as described herein, is capable of sterilizing one, two or three handpieces simultaneously, thereby providing the ability to sterilize up to about twelve sterilized handpieces per hour (with three handpieces in each cylindrical housing 50 using tubular insert 58 as described below).

A further object of some embodiments relates to providing an improved sterilization unit in which both the sterilization process and the cooling process are performed in a single unit and in a relatively short time, so as to allow sterilization of the handpieces between patients without substantial delay, that is inter-patient sterilization. In some embodiments, the unit is sufficiently small as to be able to be placed into the dental operatory itself if desired, thereby avoiding the need for a separate sterilization area or alcove.

These and other features and advantages of various embodiments of the present invention will be understood upon consideration of the following detailed description of the invention and the accompanying drawings.

### BRIEF DESCRIPTION OF THE DRAWINGS

To facilitate understanding, identical reference numerals have been used, where possible, to designate identical elements that are common to the figures.

The drawings herein are schematic, not necessarily to scale and the relative dimensions of various elements in the drawings are not to scale. The devices and techniques of the present invention can readily be understood by considering the following detailed description in conjunction with the accompanying drawings, in which:

FIG. 1 is a perspective view of a typical sterilizing unit constructed in accordance with concepts of the present invention and which includes a heating section and a cooling section into which an elongated cartridge (also referred to as a “sterilization chamber” herein) containing the handpiece or handpieces, or other surgical instrument(s), to be sterilized is successively inserted during the sterilization cycle. FIG. 1 has been taken from, U.S. Pat. No. 5,520,892 (“'892”). Although the general appearance is not substantially different from '892, the internal workings are quite distinct, as described in detail below.

FIG. 1A is a rear view of the unit of FIG. 1, also from '892 and subject to the same limitations as described in connection with FIG. 1.

FIG. 2 is a longitudinal sectional view taken along the lines 2-2 of FIG. 1 and showing the heating section of the sterilizing unit. Although FIG. 1 derives from '892, the inner components as depicted in FIG. 2 and discussed below are distinct from '892.

FIG. 3 is a longitudinal sectional view taken along the lines 3-3 of FIG. 1 and showing the cooling section along with the temperature controller and solid-state relay switch of the sterilizing unit. Although FIG. 1 derives from '892, the inner components as depicted in FIG. 3 and discussed below are distinct from '892.

FIG. 4 is an exploded perspective depiction of a typical elongated cartridge for insertion into the heating section and cooling section of the sterilizing unit depicted in FIG. 1. FIG. 4 has been taken from '892. Although the general appearance is not substantially different from '892, the internal workings are quite distinct, as described in detail below.

FIG. 5 is an exploded perspective view of the elongated cartridge, viewed from the opposite end of the cartridge from the perspective depicted in FIG. 4. FIG. 5 has been taken from '892. Although the general appearance is not substantially different from '892, the internal workings are quite distinct, as described in detail below.

FIG. 6 (on the same drawing sheet with FIG. 1) is a side view of the outside of the elongated cartridge 50 in assembled form and ready to be inserted into the heating section 12 of the sterilizing unit of FIG. 1. FIG. 6 is also from '892 and subject to the same limitations as described in connection with FIG. 1. FIG. 7 is a side sectional view of the elongated cartridge 50 of FIG. 6 containing a handpiece inserted into the heating section of the sterilizing unit of FIG. 1.

FIG. 7 is a side sectional view of the elongated cartridge 50 of FIG. 6 containing a handpiece inserted into the heating section of the sterilizing unit of FIG. 1. FIG. 7 is also from '892 and subject to the same limitations as described in connection with FIGS. 1 and 6.

A verbatim extract from a concurrent FDA submission for the present device is contained in the Annex hereto and incorporated herein by reference for all purposes. The Annex retains the same page numbers as on the FDA submission.

Table II also contains an Annex FIG. 7 at Annex page 179 and a reference thereto on Annex page 177. No reference other than that on Annex page 177 of Table II relate to this Annex FIG. 7 on Annex page 179 of Table

FIG. 8 is a schematic circuit diagram depicting a typical circuit pursuant to some embodiments of the present invention for energizing heating elements in heat-up mode in the heating section of the sterilizing unit of FIG. 1, and for activating cool-down mode in the cooling section.

FIG. 9 is a graphical depiction of the relationship of temperature and time in the heating section beginning when the unit is first electrically energized (Time=0 min.) to when steady state sterilization temperature is first achieved (at a typical Temp=134 deg. C).

FIG. 10 is a schematic circuit diagram of a typical timing circuit which is included in the elongated cartridge 50 of FIGS. 4, 5 and 6.

FIG. 11 (on the same sheet as FIG. 9) is a graphical depiction of the relationship between temperature and time in the heating and cooling sections of the sterilizing unit of FIG. 1.

FIG. 12 is a graphical depiction of the relationship between temperature, pressure and time for the entire sterilization process in the sterilization chamber of the sterilizing unit of FIG. 1. This figure illustrates the temperature and pressure rise and maintenance at steady state sterilization conditions for the duration of the sterilization process and beyond. This illustrates the ability of the device to maintain its pressure-temperature relationship for an extended length of time (if so desired) from the Lime at which the unit is first electrically energized.

### BRIEF DESCRIPTION OF DRAWINGS IN THE ANNEX

The Annex appended hereto is a verbatim extract from the present inventor's submission to the FDA, K140736. The page numbers and figure numbers from that submission have been retained in this Annex. This Annex is incorporated herein and made a part hereof for all purposes. The following Brief Description of Drawings refers to this Annex.

Annex p. 162: Cold spot mapping, thermocouple lead configuration, single handpiece.

Annex p. 163: Cold spot mapping, thermocouple lead configuration, double handpiece.

Annex p. 164: Cold spot mapping, thermocouple lead configuration, triple handpiece.

Annex p. 165-166: Cold spot mapping data for a single handpiece load.

Annex p. 167: Graphical depiction of cold spot mapping for a single handpiece load.

Annex p. 168: Graphical depiction of cold spot mapping for a single handpiece load beginning at 22 minutes and having an expanded temperature scale.

Annex p. 169-170: Cold spot mapping data for a double handpiece load.

Annex p. 171: Graphical depiction of cold spot mapping for a double handpiece load.

Annex p. 172: Graphical depiction of cold spot mapping for a double handpiece load beginning at 22 minutes and having an expanded temperature scale.

Annex p. 173-174: Cold spot mapping data for a triple handpiece load.

Annex p. 175: Graphical depiction of cold spot mapping for a triple handpiece load.

Annex p. 176: Graphical depiction of cold spot mapping for a triple handpiece load beginning at 22 minutes and having an expanded temperature scale.

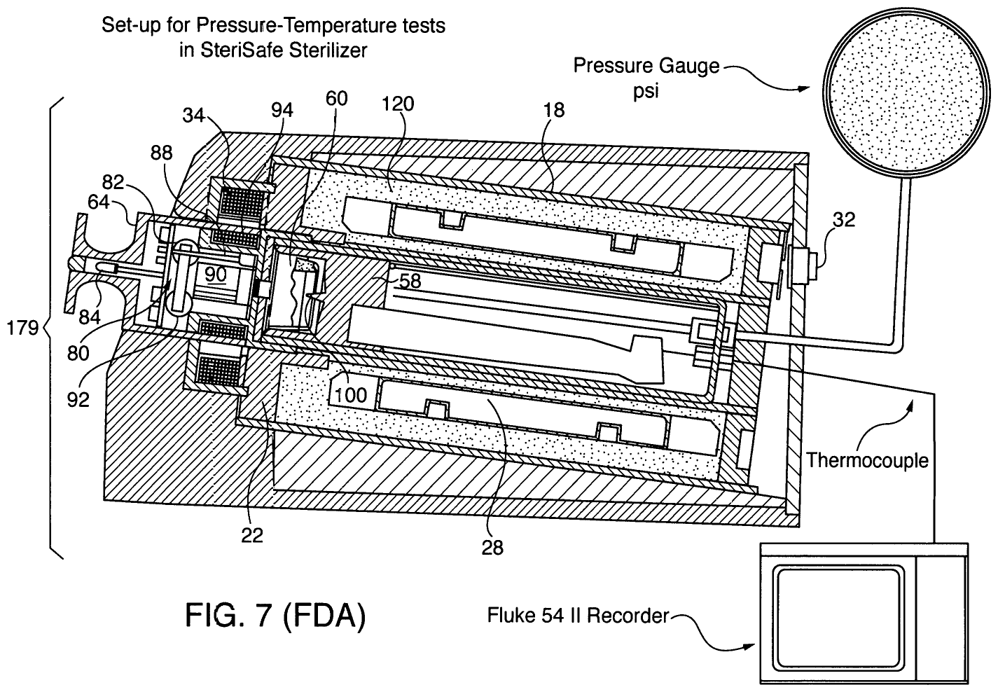

Annex p. 179, Annex FIG. 7: Set-up for pressure-temperature tests for a typical embodiment of the present sterilizer, denoted “SteriSafe HP Sterilizer™”.

Annex p. 180-181: Pressure-temperature measurement for a typical embodiment of the sterilizer described herein, as filed with the FDA for the single handpiece load.

Annex p. 182: Graphical depiction of temperature-pressure data for a single handpiece from Test Run 1 of Annex p. 180-181. The numerical values on the vertical axis denote both the temperature in Deg. C as well as the pressure in psi.

Annex p. 183: Graphical depiction of temperature-pressure data for a single handpiece load from Test Run 2 of Annex p. 180-181. The numerical values on the vertical axis denote both the temperature in Deg. C as well as the pressure in psi.

Annex p. 184: Graphical depiction of temperature-pressure data for a single handpiece load from Test Run 3 of Annex p. 180-181. The numerical values on the vertical axis denote both the temperature in Deg. C as well as the pressure in psi.

Annex p. 185-186: Pressure-temperature measurement for typical embodiment of sterilizer described herein with triple handpiece load.

Annex p. 187: Graphical depiction of temperature-pressure data for a triple handpiece, Test Run 1 of Annex p. 185-186. The numerical values on the vertical axis denote both the temperature in Deg. C as well as the pressure in psi.

Annex p. 188: Graphical depiction of temperature-pressure data for a triple handpiece, Test Run 2 of Annex p. 185-186. The numerical values on the vertical axis denote both the temperature in Deg. C as well as the pressure in psi.

Annex p. 189: Graphical depiction of temperature-pressure data for a triple handpiece, Test Run 3 of Annex p. 185-186. The numerical values on the vertical axis denote both the temperature in Deg. C as well as the pressure in psi.

Annex p. 190: Pressure measurement test data when the temperature at the head of the handpiece is 133 deg. C. for single, double and triple handpiece loading.

### DETAILED DESCRIPTION

After considering the following description, those skilled in the art will clearly realize that the teachings of the invention can be readily utilized in the sterilization of medical, dental and related handpieces and similar instruments.

The sterilization units and methods described herein are an important improvement on the work of Bowen described in U.S. Pat. No. 5,520,892, (“Bowen”), the entire contents of which is incorporated herein by reference for all purposes. In summary, the major improvements described herein result in more reliable, more thorough and/or more rapid sterilization by employing superior means of temperature control. Reliable and thorough sterilization are critical factors in obtaining FDA approval to market such a device in the US, clear evidence of which is the FDA approval to market obtained by exemplary devices described and included in the disclosure and absence of such approval for Bowen's device.

In addition, rapid sterilization is a desirable feature particularly in smaller medical and dental offices so handpieces and instruments are promptly available for use and not expending unproductive time while devices await sterilization.

The improvements herein over Bowen fall generally into three broad categories:

(1) Improved temperature control and sensing including a feedback system of control, improved temperature sensing with improved location of temperature sensors and heating elements, and improved methods and components for handling temperature data and controlling heating elements.

(2) Independent measurement of pressure. Sterilization devices measuring only temperature, with or without a temperature feedback loop may not be producing saturated steam if the pressure is not indicative of saturated steam. Saturated steam relates to the interface between liquid water and steam, a curved line on the Pressure-Temperature phase diagram of water. Knowing only the temperature locates the system along a horizontal line of constant temperature. The pressure is also needed to determine if saturated steam is present or not. Failure to sense the pressure causes serious uncertainty as to whether or not saturated steam is present.

(3) Various other components and materials are selected to provide effective heat transfer as described in more detail elsewhere herein.

To provide a concrete description, we focus our discussion on dental handpieces or instruments. These instruments are expected to provide an important practical application for the technologies disclosed herein, but these typical examples permit clear modifications and generalizations for other instruments as would be apparent to one with ordinary skills in the art. For economy of language, we use the term “handpiece(s)” and/or “instrument(s)” interchangeably, understanding thereby that we are not limited to dental handpieces but intend any dental, medical, surgical, cosmetic or other instrument for which sterilization from possible biological contamination is desired.

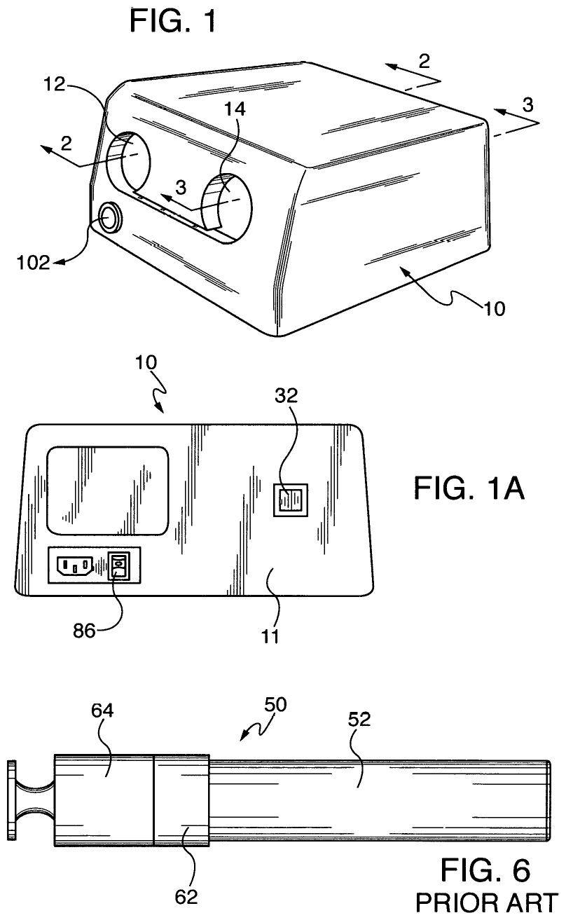

A description of the sterilization unit and its operation can be given with reference to the drawings. A sterilizing unit constructed in accordance with some embodiments of the invention is shown in the frontal perspective view of FIG. 1. The sterilizing unit is designated 10. It has typically two apertures 12 and 14 in its front face. The aperture 12 receives an elongated cartridge 50 into the healing section of the sterilizing unit of FIG. 1, and the aperture 14 receives the elongated cartridge 50 into the cooling section. To perform the sterilization operation, the elongated cartridge 50 is first inserted into the heating section through aperture 12, and then after a predetermined period of time under appropriate processing conditions, the cartridge 50 is withdrawn from the heating section and inserted through aperture 14 into the cooling section for a second predetermined time and cooling protocol. The cartridge 50 is then removed from the cooling section, opened, and the sterilized handpiece or handpieces or other surgical instrument or instruments are removed.

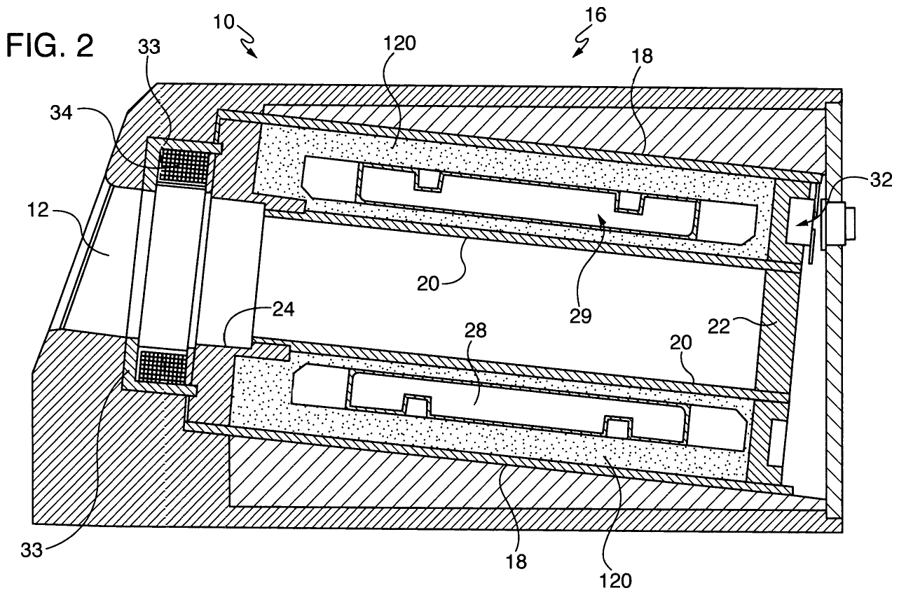

The heating section of the sterilizing unit 10 of FIG. 1 is shown in the sectional view of FIG. 2, the heating section of unit 10 designated as 16 located behind aperture 12. As depicted, the heating section includes an outer tubular case (“outer case”) 18, and an inner tubular case (“inner case”) 20. Inner and outer cases 18 and 20 are typically made of aluminum or tubular aluminum, but other materials can be used that have suitable strength and thermal conductivity properties. Inner case 20 is supported coaxially within the outer case as depicted. The outer and inner cases 18 and 20 are held in an assembled condition by back end wall 22 and forward end wall 24, and the resulting structure is mounted within the sterilizing unit 10 coaxially aligned with respect to the aperture 12. The forward end wall 24 has an annular form so that the elongated cartridge 50 may be inserted through the aperture 12 and into the inner tubular case 20 in coaxial relationship therewith.

Safety from the high temperatures used in the present sterilizer is always a concern. The housing of unit 10 is conveniently formed of molded urethane, or other heat insulating material. It is found that a polyurethane material such as BAYDUR® formed into the housing by reaction injection molding is an advantageous method of producing the housing. The space between the wall of unit 10 and outer case 18 is filled with appropriate heat insulating foam or other insulator. A muffler wrap material that can withstand 2000 deg. F and a heat shield material rated to 550 deg. F are advantageously employed.

Electric heaters 28 and 29 are mounted within the annular space between the inner case 20 and outer case 18. A thermocouple 51 is mounted onto the outer wall or the weldment of the aluminum case and it is electrically connected to a temperature controller (“controller”) 49 which is in turn connected to a relay switch (typically a solid state relay switch) 48 which is connected to the power supply for supplying power to the electric heaters 28 and 29.

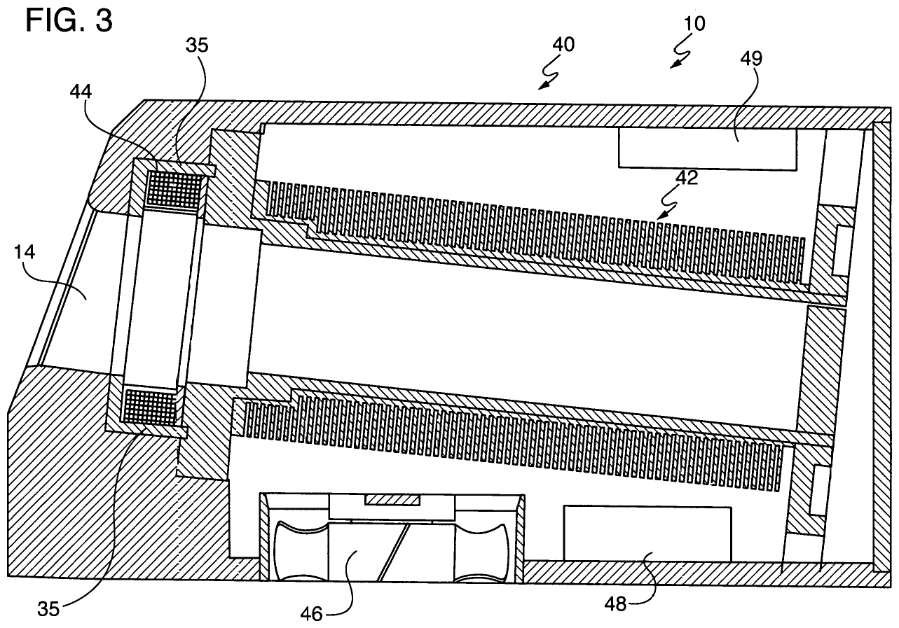

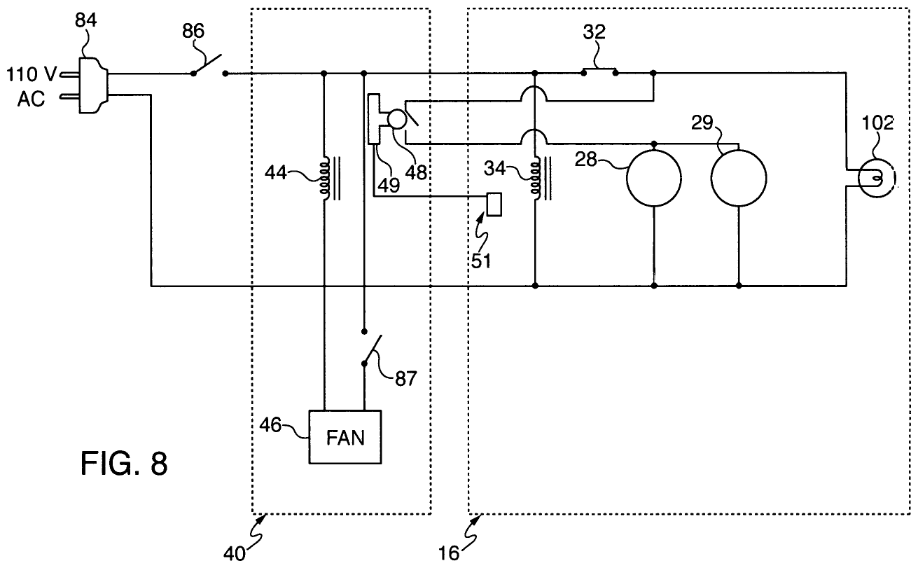

A thermocouple or other temperature-sensing device is depicted as 51 in FIG. 8. Physically, thermocouple 51 is typically located slightly off center so would not be depicted in longitudinal cross-sectional FIGS. 2, 3, 7. However, since FIG. 3 depicts the cooling unit, ordinary there is no need for a temperature sensor in unit 3 so no thermocouple is generally present. In the depictions of the heating unit 2, 7, the thermocouple typically is located slightly off center (and not depicted in the Figures) at the bottom distal one-third of the outer case 18.

One embodiment of this control system either interrupts power to the heater or allows power to the heater depending upon the temperature recognized or sensed by the thermocouple. Other embodiments can allow for gradations of current to be supplied to the heater, such as with a rheostat, as would be apparent to one having ordinary skills in the art of temperature control.

A thermal switch 32 is typically mounted in or on the back end wall 22 adjacent the end of the annular space. The annular space between the inner and outer cases 20 and 18 is filled with a material functioning as thermal ballast, 120, typically paraffin, which is sealed into the annular space, the electric heaters 28, 29 being immersed in the thermal ballast or paraffin wax. An induction coil 34 is also mounted in the sterilizing unit to surround the aperture 12, as shown in FIG. 2. A magnetic core 33 surrounds the induction coil 34, which also acts as a magnetic shield. This core can be steel or, to reduce the chances of overheating, other materials as described elsewhere herein.

In the cooling section of the sterilizing unit surrounding aperture 14, a similar magnetic core 35, which also acts as a magnetic shield, surrounds the induction coil 44, as depicted in FIG. 3.

In some embodiments of the sterilizer, for some choices of the steel core 33, overheating is a concern. It is advantageous to choose materials for the core not prone to overheating under the operating conditions of the sterilizer. Polyetherimide plastics are found to be one suitable choice. In particular, DURATRON® by Quadrant Engineered Plastic Products, and ULTEM® by SABIC (Saudi Basic Industries Corporation) are found to be suitable commercial products for this purpose. It is likewise often advantageous to employ a polyetherimide plastic instead of a steel magnetic core anyplace in the sterilizer in which heating or overheating is a concern.

It is often prudent as a safety precaution in the case of overheating to employ at least one additional thermal switch in series with switch 32.

It is an advantageous safety feature to provide an indicator such as a light on cartridge 50 (or similar warning conspicuous to the operator) to illuminate and inform the operator that the cartridge is hot and should be handled with care when an elevated temperature is present. It is also advantageous that this light be battery powered with on-board batteries on cartridge 50 so that a hot cartridge does not lose its high temperature indicator when it is removed from the electrical power of the heating unit for transfer to the cooling unit. This may be done in some embodiments of the present invention by using the magnetic induction voltage induced by the current flow within the heating unit to charge the on-board batteries of the cartridge with every use, thus maintaining continuous high-temperature warning without concern for dead on-board batteries.

The cooling section of the sterilizing unit 10 of FIG. 1 is designated 40 in FIG. 3, and it includes a finned tube 42 mounted within the unit 10 in essentially coaxial relationship with the aperture 14. Tube 42 is suspended on the front and back of unit 10. An induction coil 44 surrounds the aperture 14, as shown, and it is surrounded by a magnetic core, which also acts as a magnetic shield, 35. Advantageously, a fan 46 is mounted in or on the floor of the unit 10 to set up a cooling flow of air around the finned tube to cool the hot cartridge 50 when it is inserted into the finned tube at a rapid or less rapid rate depending on the rate of flow induced by fan 46. Air is thought to be the most convenient coolant for most cases but other coolants, liquid or gas, can also be used within the scope of this invention, typically circulated by a pump or fan.

A heat-insulating panel (not shown) may be mounted in unit 10 between the heating and cooling sections to improve the thermal isolation of the heating and cooling sections of unit 10. This panel may be formed, for example, of pressed glass or other suitable heat insulating material(s).

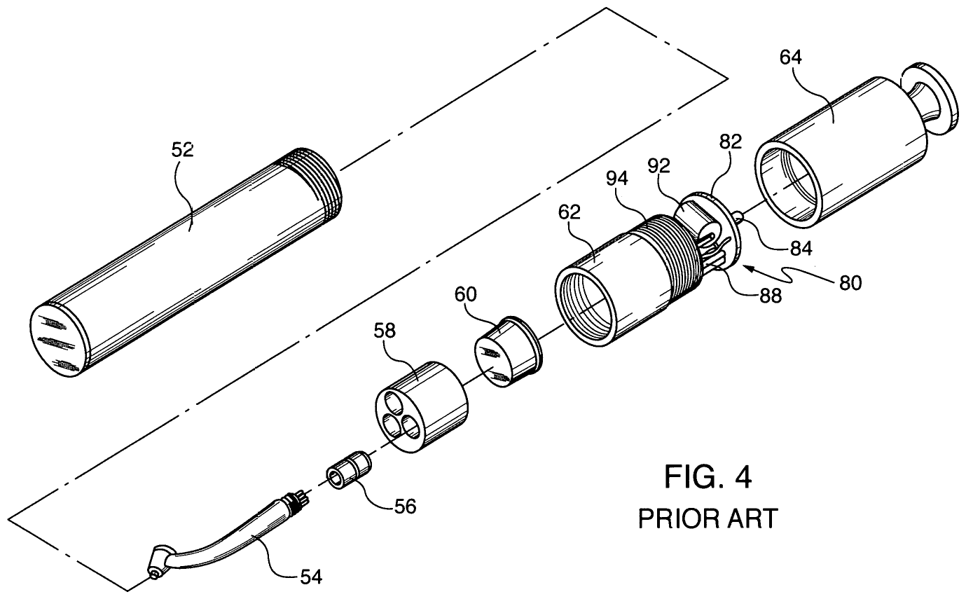

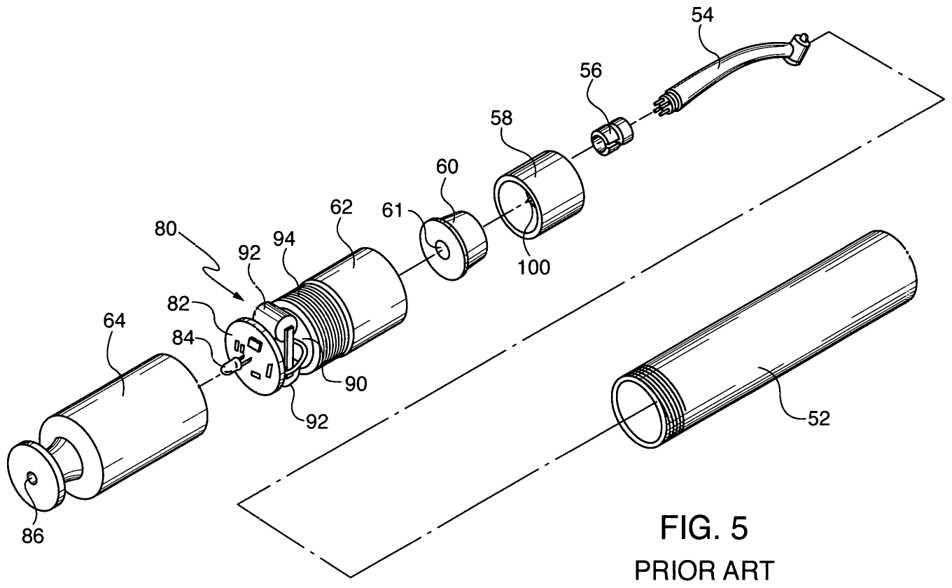

The external view of a loaded cartridge 50 as shown in FIG. 6 is depicted internally by exploded views in FIGS. 4 and 5. The exploded views depict the components of cartridge 50 as a loaded sterilization chamber containing (for illustration, not limitation) a single handpiece. The operator need only thread the cylindrical housing (“housing”) 52 onto cap 62 to assemble the sterilization chamber prior to inserting it into the aperture 12 for sterilization. The cylindrical housing 52 is open at one end and closed at the other end. The cartridge 50 shown in FIG. 6 is shown in exploded views in FIGS. 4, 5 including a cylindrical housing 52, open at one end and closed at the other end. The housing 52 receives at least one handpiece 54, or other surgical instrument(s) or articles to be sterilized. As described below, some embodiments permit multiple handpieces to be contained in a single housing and sterilized concurrently. However, for economy of language we describe the detailed operation of the device as if only a single handpiece were undergoing sterilization.

The handpiece is inserted into the cylindrical housing 52 through its open end. A tubular adaptor (“adaptor”) 56 is threaded or fitted to one end of the handpiece 54, and it is received in a tubular insert (“insert”) 58 in a press fit with a channel in the insert. The insert 58 is advantageously constructed so that up to three handpieces 54 may be supported for simultaneous sterilization. Larger versions of this device, or smaller handpieces to be sterilized, may allow for more than three handpieces to undergo concurrent sterilization. Such modifications would be apparent to those having ordinary skills in the art and are included within the scope of the present disclosure.

The insert 58 has a well, formed in the opposite end from the end receiving the handpiece(s), which receives a water ampoule 60. A cap 62 is fitted over the insert and threaded onto the end of the cylindrical housing 52. The cap 62 includes electronic circuitry, as described below, which performs a timing and control function. A cover 64 is fitted over the cap 62 and threaded onto the end of cap 62. A spring retainer is advantageously mounted in the end of cylindrical housing 52 to hold insert 58 in place when the cap 62 is threaded to the end of cylindrical housing 52 to complete the loaded cartridge (or the “sterilization chamber”).

To be concrete in our description, we use two heaters or heating elements. This is for illustration, not limitation, since any convenient number of heaters can be employed within the scope of this invention.

Electrical heating elements 28, 29 are shown in the circuit diagram of FIG. 8. Heating elements 28 and 29 are typically positive temperature coefficient (PTC) heating elements although other types of heating elements are not excluded. The PTC heating element 28 is selected to have a Curie point of about 150° Centigrade (“C”), and the heating element 29 is selected to have a Curie point of about 190° C. The circuit is intended to plug into the usual 110 volt AC receptacle through plug 84, although it can be easily adapted for use with other voltages. One contact of plug 84 is connected through a manual power on-off switch 86 and through a normally closed, manually reset, thermal overload switch 32 to PTC heating elements 28, 29. Switch 32 is selected so as to open when the thermal ballast (for example, paraffin wax) surrounding the heating section reaches a temperature of 146° C. This temperature represents an overload temperature, and when switch 32 opens, it de-energizes the system, and this switch stays open until it is manually reset at the back panel 11 as shown in FIG. 1A. Manual power switch 86 is also located on the back panel 11 shown in FIG. 1A. The other contact of plug 84 is directly connected to the other side of the PTC heating elements 28 and 29 and feedback loop which contains the solid-state switch 48. The manual power switch 86 is also connected through the normally closed thermal switch 32 and through to one side of the PTC heating elements 28, 29, via solid state relay switch 48, the other side of which is returned to the other contact of plug 84.

The feedback loop advantageously employed herein is designed so that the thermocouple 51 relays the temperature of the outer aluminum case 18 to the temperature controller 49. When the temperature of the outer aluminum case reaches a temperature indicating that a temperature of 133° C. has been obtained at the head of the handpiece 54 located within the sterilization chamber cartridge 50 (as determined by prior system calibration), the temperature controller 49 sends a signal to the solid state relay switch 48 to interrupt (or reduce) the power to the heaters 28 and 29. This typically results in a slight temperature overshoot and when the handpiece temperature drops back to 133° C. the solid-state relay switch again energizes the electric heaters. In so doing the temperature is maintained at 134° C.±1° C. at steady state within the cartridge 50 for the entire period of the sterilization process. A power-on indicator lamp 102 may be located on the front panel of the unit (FIG. 1) and connected through switches 32 and 86 across the contacts of plug 84. Induction coil 44 is located in the cooling section 40, and this induction coil is connected across the contacts of switch 84 through the on/off switch 86. The induction coil 34 in the heating section is also connected across the contacts of plug 84 through the manually operated on/off switch 86.

The present system has been designed so as to bring the temperature at the head of the handpiece(s) to 134 degrees C.±1 degree. It is calibrated to produce that temperature by measuring the temperature at a point on the outside of the wax containment housing which produces the desired temperature at the head of the handpiece(s) and steady state conditions in the sterilization chamber. Maintaining the temperature at the wax containment housing enables the temperature to be maintained in the sterilization chamber due to the close proximity (of the external sterilization cylinder and the internal wall of the wax chamber 20, typically about 0.001 inch.

The Annex hereto, including Table I and Table II is incorporated herein by reference and made a part hereof for all purposes. The Annex is an excerpt from an FDA 510(k) filing by the inventor in connection with the devices disclosed herein and retains the page numbers from the FDA submission. Table I gives the test results of single, double and triple handpiece loads. These results show that the head of the handpiece is the “cold spot” of the sterilization chamber and also provides calibration information for deriving the temperature at the cold spot from temperature readings elsewhere in the sterilizer.

This is to be contrasted with Bowen '892 that does not disclose a calibration step but rather only describes the temperature inside the chamber. This omission can lead to “cold spots,” incomplete sterilization and potential difficulties in obtaining FDA approval for the device.

When the sterilizing unit is first turned on by closing the on/off switch 86, both PTC heating elements 28 and 29 are connected in parallel across the AC source, and the heating section 16 of the unit rapidly heats up to operating temperature. When the thermal ballast temperature reaches a temperature, which corresponds to a temperature of 133° C. within the cartridge 50, the feedback loop goes into operation. The feedback loop maintains the temperature within the cartridge 50 at a temperature of 134° C.±1° C. for the entire sterilization process. This temperature is maintained by the feedback loop allowing or interrupting electrical energy to the heating elements as is necessary to maintain said temperature in the cartridge 50. The feedback loop in some embodiments advantageously consists of a thermocouple 51 connected to a temperature controller 49, which is connected to a solid-state relay switch 48 which is connected to the heating elements. It is normal practice to keep the on/off switch 86 closed during the course of the working day when frequent use of the sterilizing unit is anticipated.

When the cold cartridge 50 (that is, substantially at room temperature) is inserted into the heating section 16, it causes the temperature of the thermal ballast to drop. This temperature drop causes the thermocouple 51 to send a signal to temperature controller 49, which closes the solid-state relay switch 48, which energizes heaters 28 and 29. This action enables the ballast (typically wax) to be rapidly returned to its operating temperature, heating the interior of the cartridge 50 to a temperature of 134° C.±1° C. Whenever the on/off switch 86 is closed, the indicator lamp 102 is energized indicating that the unit is on.

When the cooling section 40 is at room temperature, the thermal switch 87 is open and the fan 46 is de-energized. However, when the hot cartridge 50 is removed from the heating section 16 and inserted into the cooling section 40, its heat causes the thermal switch 87 to close and operate the fan. The fan continues to operate until the temperature of the cartridge 50 within the cooling chamber is returned to room temperature, or until the cartridge 50 has been removed and the interior of the cooling section returns to room temperature.

As described in U.S. Pat. No. 4,734,560 (“'560”, the entire contents of which is incorporated herein by reference), the PTC heating element is well known. The PTC heating element is typically composed of a semi-conductor ceramic, such as an appropriately doped barium titanate. This material has a positive thermal coefficient, and it has a property that at a certain temperature, known as the Curie point, its internal resistance suddenly increases if temperatures are raised above that point.

It is important to note that '560 depends on the latent heat of fusion of the paraffin wax to establish a precise sterilizing temperature. The embodiments of the present invention employ other, more reliable and robust structures and means of temperature control as described elsewhere herein.

Accordingly, the PTC constitutes an advantageous heating element because of its automatic temperature control. The PTC heating element is independent of voltage, and it can be used in connection with alternating current. Regardless of voltage, the element will increase in temperature until the Curie point is reached, and at that point it will effectively cut off, serving inherently as an automatic temperature controller. Moreover, the PTC heating element does not require a protective relay in its circuit, because it is incapable of burning out. The Curie point of the PTC heating element can be set to any desired temperature level by controlling the doping of the ceramic material. In the case of the sterilizer unit of the present invention, the temperature level is set to a particular value, as will be described. The aforesaid temperature-controlling loop adds to the ability to control temperature accurately. Although the PTC heating element is advantageously used in some embodiments of the present invention, other types of heating elements can also be used effectively due to the precise temperature control enabled by embodiments of the feedback loop system described herein.

It is known that the Curie point in a PTC heating element cannot be set precisely and variations of up to ±40% have been experienced from one PTC heating element to another. However, in the sterilizing units described herein pursuant to some embodiments of the present invention, the PTC heating elements 28 and 29 are embedded in (typically) a paraffin wax thermal ballast, as described above, and the wax functions as a medium to carry the heat from the heating elements to the interior of the heating section of the unit. The paraffin wax is selected to have a melting point which corresponds with a high degree of accuracy to the desired temperature in the sterilizing unit. The Curie point of the PTC heating element 28 is then set to occur above the desired temperature, even allowing for its widest variation. Although the heating elements are advantageously chosen to be of the PTC type, this is not a firm requirement as the feedback temperature control system described herein functions satisfactorily with other types of heating elements. Accordingly the sterilizing temperature may be regulated to within about ±1° C., and to have a temperature reproducibility of about ±1° C.

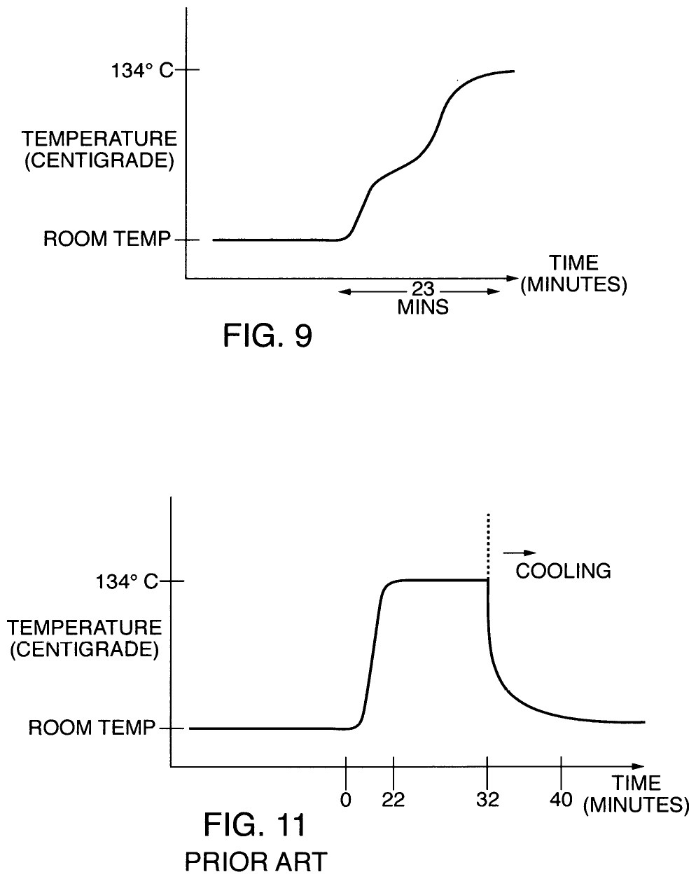

The sterilizing unit in some embodiments described herein has an added feature of rapid heat-up of the heating section. Accordingly, when the sterilizer unit is first turned on from room temperature, both heating elements 28 and 29 operate together to rapidly bring the paraffin wax up to a temperature which corresponds to a temperature of 133° C. in the sterilization chamber. When that temperature is reached, the feedback loop begins its temperature control by switching on and off heating elements 28 and 29 and the paraffin wax is maintained at an operating temperature which corresponds to a cartridge temperature of 134° C.±1° C. Accordingly, the temperature of the heating section follows the curve of FIG. 9 after the unit is first turned on, with the temperature being raised rapidly from room temperature to 134° C.±1° C., at which time the paraffin wax is maintained at a constant temperature by PTC heater element 28 and 29 working in concert with the feedback loop. Switch 32 is an overload switch and it stays closed throughout the sterilizing process, unless an overload condition occurs. If an overload condition arises, switch 32 opens and typically must be reset manually.

To perform a sterilization procedure the elements of the cartridge 50 shown in FIGS. 4 and 5 are assembled and placed into the cylindrical housing 52. Specifically, the adapter 56 is screwed onto or press fit onto the end of handpiece 54 and the combined handpiece and adapter are manually press fit into a friction fit channel in the insert 58. The insert 58 and attached handpiece 54 are then inserted (suspended) into the cylindrical housing 52. The water ampoule 60 is inserted into the other end of insert 58, the well. The cap 62 is then placed over the insert 58 and screwed onto the end of the cylindrical housing 52. When that occurs, a barb 100 located in the well of insert 58 pierces the water ampoule 60 so that water from the ampoule in the form of liquid, steam, and saturated steam can travel down through the insert and through the internal tubing of the handpiece 54 effectively flushing and sterilizing handpiece 54.

It is advantageous to use an insert 58 having the proper number of openings (or channels) for the number of handpieces to be sterilized. For example, an insert suited for three handpieces should not be used to sterilize one or two handpieces with the other mounting channel(s) left empty. This configuration leads to “dead-end” regions in the insert at the bottom of the unoccupied channels which are difficult to flush and sterilize. Advantageously, an insert should be used in which all channels are occupied by a handpiece.

The water-containing ampoule 60 is formed, for example, of polystyrene, and it becomes gradually flattened by heat and pressure during the heating cycle so that water in the ampoule is slowly dispensed to flush the handpiece and then to be converted to steam and saturated steam. The dispensing of the water from the ampoule continues until the ampoule becomes completely flattened. When the cartridge 50 is placed in the healing unit, the water tram the ampoule is converted to steam and saturated steam. The geometry of the cartridge 50 assures a homogeneous and isotropic mixture of air and saturated steam during the steady state sterilization process. When the cartridge 50 is placed in the accurately temperature and pressure controlled heating section of the sterilizing unit, the conversion of water to saturated steam produces a temperature and pressure that are dependently related and follow conventional saturated steam tables, such as that of the American Society of Mechanical Engineers (ASME). Such a relationship of temperature and saturated steam pressure correlated to time at steady state conditions is generally accepted in the field as a condition for using saturated steam as a sterilant that produces sterilization conditions, that is:

121° C. at 15 psi for 20 minutes

128° C. at 38 psi for 10 minutes

134° C. at 45 psi for 3.5 minutes

Accurate control of the temperature within the cartridge 50 is necessary to prevent rupture of the cartridge 50 and to prevent damage to the instruments being sterilized in the sterilization cycle.

Accordingly, the water ampoule 60 is placed in the enclosed cartridge 50 in such a manner as to force water, steam and saturated steam through the tubing and channels of the handpiece to flush debris and biological contaminants from the instrument. The flushing process also ensures that all parts of the handpiece are contacted by steam and saturated steam to sterilize the internal and external parts of the handpiece.

The water in the water ampoule 60 is advantageously ultrapure water, typically as previously processed by ion exchange, distillation or reverse osmosis and filtration or a combination of these water purification methods. Such water also performs a de-scaling operation of the instruments being sterilized. Advantageously, such water has a specific resistance greater than about 5,000,000 ohm-cm. Attached to the top of the ampoule may be a color-change chemical indicator-integrator which serves to indicate to the operator whether or not the sterilization process has been completed and sterilization conditions have been met. When sufficient time has elapsed with the proper temperature, pressure, and saturated steam conditions to ensure that the conditions for sterilization have been met the chemical integrator-indicator will change color. At the end of the sterilization cycle, and when the cartridge 50 is disassembled, the chemical indicator on the flattened ampoule will indicate by a change in color whether or not the instrument has been exposed to sterilization conditions. The chemical indicator-integrator may be of the type commercially manufactured and marketed, for example, by Albert Browne Ltd. of Leicester, United Kingdom or the Steris Corporation or other manufacturer. The color indicator comes in the form of a dot that has a particular color at the beginning of the process, and it assumes a selected colored only when the sterilization process has been completed and the necessary time, temperature, pressure and saturated steam criteria have been achieved.

Other liquids may be contained in the ampoule, such as, a lubricant which additionally serves to lubricate the handpiece; or disinfecting chemicals, such as alcohol, formaldehyde, or peroxide and the like, which may permit reductions in the sterilization times and temperatures. Dyes may be added to reveal the quality or quantity of flushing that occurred.

The circuitry in the cap 62 assures that the sterilization process in the heating section of the unit will have a proper time duration, this being achieved by a pressure switch 90 which measures the pressure in the cartridge 50 to control the process time, this being a more accurate basis for sterilization than measuring temperature in the cartridge 50. Advantageously, in some embodiments temperature can also be measured along with pressure by adding a temperature gauge to the embodiments described herein. Such concurrent monitoring of both temperature and pressure produces a more reliable indication whenever a sterilization process has failed.

That is, for saturated steam, temperature and pressure are related so that knowing either implies a value for the other. But if some problem has occurred in the sterilization process so that non-saturated steam is present, this would be detected by knowing both temperature and pressure. Knowledge of only temperature or pressure does not ensure that saturated steam has been achieved. Therefore, concurrent knowledge of both pressure and temperature provides an important check on the presence (or not) of saturated steam. Table II provides the results of tests submitted to the US FDA concerning temperature-pressure tests for some embodiments of the present invention.

The present approach to sterilization should be contrasted with that of Johansen (US Patent Application Publication No. 2002/0068029 A1) in which hot steam of an undetermined temperature is supplied from an external autoclave to a chamber containing one or more instruments to be sterilized. When the temperature/pressure in the chamber reach preset values, two outlet valves are opened and closed in an alternating manner ¶ [0034]. No mention is made of striving for the production of saturated steam. Producing saturated steam and holding in a steady state for a period of time is not feasible with the device of Johansen, thereby precluding timed exposures of the instruments to saturated steam. As discussed elsewhere herein, temperatures well in excess of saturated steam maybe easily obtained with an autoclave, but these are damaging to many instruments and must be avoided. Johansen shows no way to do that, unlike the present disclosure.

Thus, the pressure switch 90 has an important function in ensuring adequate sterilization. The pressure switch is selected to coordinate with the temperature at which the production of saturated steam under steady state conditions occurs in the cartridge 50. In this manner the beginning of the timing function of the timing circuitry corresponds to the time at which the sterilization process begins. In some embodiments of the present device, then, the timing initiated by the activation of the pressure switch corresponds to the beginning of the sterilization process and the total time for the sterilization process is selected by the proper selection of the timing circuitry.

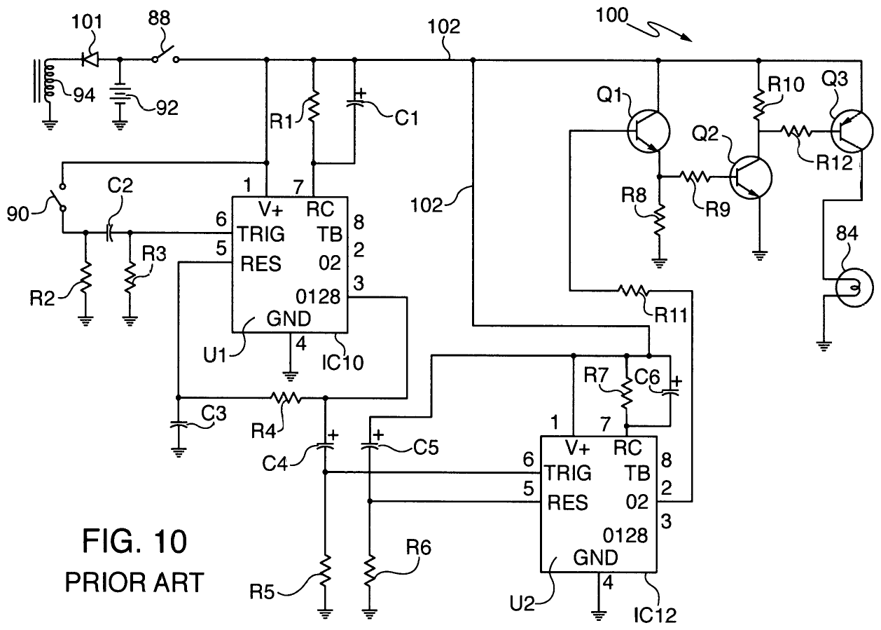

The timing-logic control module 80, as shown in FIG. 7, is contained in cap 62. The module includes a printed circuit board 82 on which the electrical elements of FIG. 10 are mounted. The printed circuit board also mounts an indicator lamp 84, which, when energized illuminates a lens 86 in cover 64 (FIG. 4). The printed circuit board 82 also mounts circuitry connected to a thermal switch 88. The pressure switch 90, typically located in the sterilization chamber, is also connected to the circuit in module 80, as are one or more batteries 92. As noted elsewhere, these batteries are advantageously chosen to be rechargeable while the cartridge is undergoing sterilization, but this is not an inherent limitation. Conventional disposable batteries may also be used or a direct connection with the unit's AC power source, although direct connection is contraindicated since the absence of an on-board power source causes the high temperature indicator to switch off when the unit loses its connection with AC power, typically when removed from the sterilization unit.

An induction coil 94 is mounted on cartridge 50 around the module 80, and this coil is inductively coupled to induction coil 34 when the cartridge 50 is inserted into the heating section 16 (FIG. 2), and to induction coil 44 (FIG. 3) when the cartridge 50 is inserted into the cooling section.

As shown in FIG. 8, induction coil 94 is connected to a charger for (rechargeable) battery 92. Accordingly, the charger is energized whenever the cartridge 50 is inserted into the heating section, and the charger is also energized whenever the cartridge 50 is inserted into the cooling section. This assures that the batteries are maintained in a charged condition as the sterilizing unit is used. As mentioned above, fan 46 (FIG. 8) is also energized whenever the hot cartridge 50 is inserted into the cooling section.

The electrical circuitry for the timing logic control module 80 of FIG. 7 is shown in FIG. 10. The electrical circuitry of FIG. 10 includes two integrated circuits IC10 and IC12. Each of the integrated circuits is advantageously chosen to be of the type designated 7242. The Q2 output terminal of integrated circuit IC12 is connected to a buffer amplifier 100 which may comprise two NPN transistors Q1 and Q2 of the type designated 2N3904, and a PNP transistor Q3 of the type designated PN2907. The collector of the transistor Q3 is connected to one terminal of the indicator lamp 84 of FIG. 7, the other terminal of the indicator lamp is grounded. The temperature switch 88 of FIG. 7 is connected to a common lead 102 and to the positive terminal of battery 92, the negative terminal of the battery is grounded.

When the cartridge 50 of FIG. 6 is inserted into the heating section 12 (FIG. 2), the cartridge 50 begins to heat up. When a particular predetermined temperature is reached, within the cap 62 of the cartridge 50 the temperature switch 88 closes. The circuit of FIG. 10 is now energized and indicator lamp 84 is illuminated and is visible through the lens in cover 64 of the cartridge 50. It will be appreciated that the circuit of FIG. 10 will not be energized until not only the internal temperature of the cartridge 50 reaches a predetermined temperature but the temperature of all handpieces, or other instruments, which may be supported within the cartridge 50, also reaches a predetermined temperature.

Integrated circuit IC10 is connected as a timer. However, the timing interval of the timer is not initiated until the pressure within the cartridge 50 reaches a predetermined pressure of, for example 48.5 psi. From the known and tabulated properties of saturated steam, this pressure corresponds to the actual sterilizing temperature of the instruments within the cartridge 50, and is an extremely accurate measurement of the sterilization temperature as temperature and pressure are dependently related in the steady state sterilization process.

Accordingly, the circuit of FIG. 10 is activated to begin timing only after all the instruments within the cartridge 50 reach a predetermined sterilizing temperature. At that time, the pressure switch 90 opens, and the integrated circuit IC10 begins its timing function. In some embodiments, the time interval is set to ten minutes. Until the end of the timing interval is reached, the indicator lamp 84 is continuously energized.

When the end of the timing interval is reached, the output Q128 of the timer integrated circuit IC10 changes state and triggers the integrated circuit IC12 so that the indicator lamp 84 is caused to flash. At that time, the timer integrated circuit IC10 resets itself to be ready for the next operation. As mentioned above, the buffer amplifier 100 provides sufficient energy to energize the indicator lamp 84 in its continuous or flashing state.

As is also shown in FIG. 10, rechargeable battery 92 is connected through a diode 101 to induction coil 94. As shown in FIG. 8, when the cartridge 50 is inserted into the heating or cooling section of the unit 10, alternating current in induction coil 34 or 44 induces a charging current in induction coil 94 to provide a charging current for battery 92 and an instantaneous energizing potential for the electronic circuitry of FIG. 10 in the event that battery 92 has not attained its fully charged condition. Accordingly, battery 92 is maintained in a fully charged condition when the cartridge 50 is in either the heating section or the cooling section of the sterilizing unit.

In brief, when the cartridge 50 is inserted, for example, in the heating section 16 of the sterilizing unit, it is heated to the selected operating temperature. When the cartridge 50 reaches a prescribed temperature, temperature switch 88 closes and indicator lamp 84 is illuminated. This illuminated indicator lamp 84 serves to alert the operator of the sterilizing unit that the cartridge 50 is hot and thus serves as a safety monitor for the operator. The heating of the interior of the cartridge 50 continues until the internal pressure reaches 48.5 psi, this being an accurate designation that the interior of the cartridge 50 and the instruments contained therein has now reached sterilizing temperature. When that pressure is reached, pressure switch 90 opens and the integrated circuit IC10 begins its timing function. After ten minutes, the timer formed by integrated circuit IC10, times out and causes integrated circuit IC12 to send a flashing signal to the indicator lamp 84. The operator then removes the cartridge 50 from the heating section 16 and places it in the cooling section 40. The heating section of the sterilization unit 10 is now ready to receive another cartridge 50 if so desired. The indicator lamp 84 continues to flash until the pressure within the cartridge 50 contained in the cooling section, drops, for example, to 41 psi. Then pressure switch 90 closes, and the timer integrated circuit IC10 resets itself and the indicator lamp 84 returns to its continuously energized condition. The cartridge 50 is left in the cooling section 40 until the internal temperature returns to room temperature, at which time temperature switch 88 opens, and the indicator lamp 84 is extinguished, so that the cartridge 50 may now be removed from the cooling section. It should be noted that if during any operation, pressure within the cartridge 50 in the heating section is lost, the pressure switch 90 will immediately close and discontinue the operation. In this event indicator lamp 84 will not flash. This action provides a fail-safe system for the sterilization process in the sterilization unit.

It is important to appreciate that the present devices typically monitors both the pressure and the temperature independently. For example, if the pressure seal is not properly seated, it is possible for the temperature within the chamber to be correct, but the pressure is too low. With an improper pressure seal, the proper pressure is not obtained and saturated steam is not obtained even though the temperature readings will not detect a problem. If the temperature is too low, the pressure switch will not be activated. Therefore, by controlling the system via pressure, the optimum steam quality of saturated steam is assured. The absence of saturated steam causes a substandard, inadequate sterilization process and such handpieces must be re-sterilized whenever this occurs. The independent temperature and pressure monitoring described herein ensures that only properly sterilized handpieces complete the sterilization process correctly without a warning to the operator.

The sterilization units described herein respond to an internal pressure of the cartridge 50 of, for example, 48.5 psi, which correlates to a precise measurement of the actual temperature of the instruments being sterilized. The timing cycle begins only after all instruments have reached the predetermined sterilizing temperature, at which time the internal pressure is 48.5 psi, and the timing cycle begins. Accordingly, the present units are not only precise in its measurement of the sterilizing temperature through pressure, but also adjust automatically to load conditions, that is, to the size of the instruments, and to the number of the instruments within the cartridge 50. All the instruments within the cartridge 50 must reach sterilizing temperature, before the pressure will reach 48.5 psi to start the timing cycle.

Therefore, embodiments of the present invention provide a relatively inexpensive unit for sterilizing dental handpieces, and the like, which are simple to operate, which require minimum sterilization times, and which will not harm or dull the instruments being sterilized. A lubricant may be added to the liquid in the ampoule to lubricate the instruments, and/or disinfectants may be added. The entire process takes place in a sealed cartridge 50 and the instruments are not removed until the sterilization cycle has been completed. There is no venting to the atmosphere of any contaminating gases and the instruments in the cartridge 50, after sterilization, cool down without contamination.

The accuracy of the feedback loop described and used herein enables temperature control within the sterilization chamber during the sterilization process to be accurate to about plus or minus 1 degree C. This is in contrast to prior art (including the '892 patent) that depends largely upon the latent heat of fusion of the paraffin wax for temperature control. Typical PTC heaters are not accurate enough on their own to maintain temperature control to the accuracy and reliability needed to assure the physical conditions necessary to reliably sterilize the contents of the sterilization chamber. Also, the typical prior art reliance for control of the process on the latent heat of fusion may work for the first sterilization of the day but after that the wax is melted, accurate temperature control is lost. Once accurate temperature control is lost, so too is lost the control of physical conditions within the sterilization chamber.

Pressure and temperature in saturated steam are dependently related so it is not absolutely necessary to measure temperature if one measures the pressure. In fact, it is more accurate to measure pressure than temperature if only one is to be measured. If there is a leak in the system (gasket failure, improper seating, etc.) the temperature may still be maintained and appear correct if measured, but the steam quality will be compromised as the necessary pressure to guarantee saturated steam conditions according to ASME for saturated steam will be lost and so will be the guarantee of physical conditions necessary for adequate sterilization.

The timing logic module used herein typically does not begin its count until predetermined steady state saturated steam physical conditions have been achieved in the sterilization chamber (pressure, temperature, humidity, saturated steam). It is triggered at about 48.5 psi. In contrast, the '892 patent has a temperature overshoot built in as the pressure switch that activated the timing logic module was set at 26 psi and yet the temperature went to 134 degrees C. (Note that at 133 degrees C. the pressure is 48.5 psi). Therefore between the temperature attained at 26 psi and the temperature of 134 degrees C. the conditions within the sterilization chamber were not at steady state. One therefore could not guarantee the conditions in the sterilization chamber and therefore cannot guarantee reproducibility and therefore cannot reach the standard required to satisfy the FDA, and prudent best-practices requirements for sterilizers. Even if microbiological testing showed lethality in all tests, reproducibility cannot be assured.

Various embodiments of the current system are designed to prevent temperature overshoot, and the sterilization process is not triggered until saturated steam steady state conditions exist.

The temperature controller herein is adjustable so each sterilizer (device) can be adjusted for accuracy at the time of manufacture as a component of manufacturing quality control.

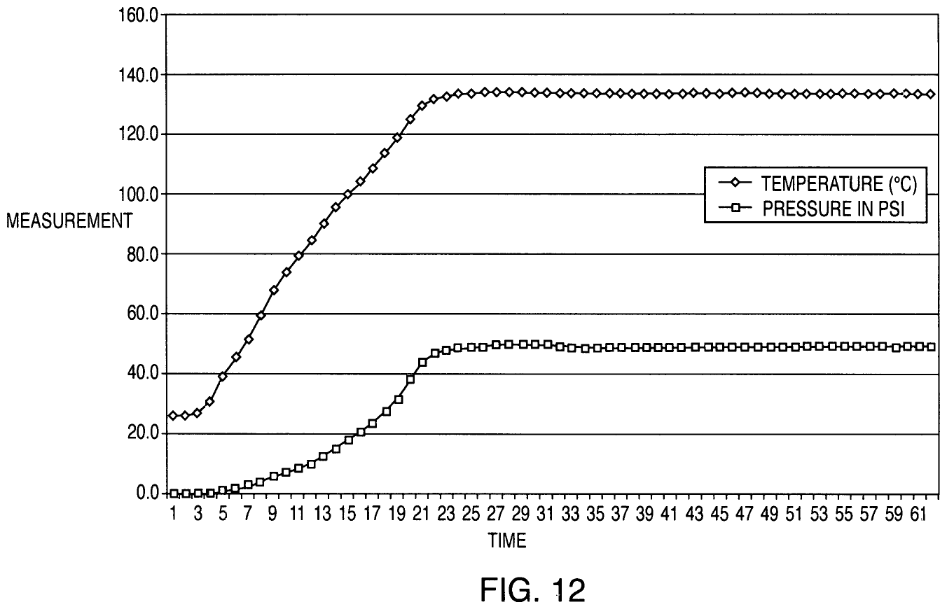

Graphs of pressure/temperature vs. time (FIG. 12) are asymptotic to 135 degrees C. (50 psi) for the dental handpiece sterilizer application. The asymptotes begin at the initiation of the sterilization process. The timing count is initiated when the pressure at the pressure switch is 48.5 psi and the temperature at the head of the handpiece is 133 degrees C. Cold spot mapping and pressure temperature tests confirm this. These tests are submitted as part of a 510(k) FDA submission and included herein as Table I. The entire FDA 510(k) submission is incorporated herein by reference for all purposes.

Some prior art including the '892 patent use PTC heaters to control temperature. But PTC's cannot be set within an accuracy of plus or minus 40% according to the '892 patent. Therefore temperature specific wax is used which has inherent drawbacks as described above.

Bagging of instruments is not necessary as required or recommended in some prior art devices. Bagging represents a possible break in the sterilization cycle and, thus, can lead to incomplete sterilization and problems obtaining FDA approval. The sterilization chamber herein is removable and the instruments can be carried to the operating theater in the sterilization chamber, thereby not risking contamination by ambient air. Also if so desired a dummy threaded plastic cap can be used to replace the sterilization cap for sterilization chamber transfer without risking contamination to the instruments as there is an insert and a collapsed ampoule between the ambient air and the sterilized instruments thereby making the purchase of several sterilization caps unnecessary. Instruments in a sterile condition can be stored safely in this fashion.

A pressure relief valve can be placed in the bottom of the cylinder if so desired to enable drying of the contents. The relief valve can be opened while the chamber is still hot to allow the hot steam to escape thereby drying the contents.

An escape vent hole can be placed in the dummy cap and a separate warmer can be used to warm the capped cylinder to drive off the water as steam into the ambient air. Alternatively, the capped cylinder can be placed in the heating section 16 to drive off the water, thereby producing dry handpieces (or other contents).

Since the temperature controller herein can be set at any desired temperature, it enables the unit to be standardized for the saturated steam sterilization conditions for any manufacturer's device to be sterilized. It enables the sterilization, for instance, of instruments that cannot withstand high temperatures to be sterilized. A lower temperature can be used for an extended period of time. (Recall that sterilization is a time-temperature-pressure-saturated steam condition phenomenon.)

Easy monitoring of the system by the operator is possible for the devices described herein. The light in the sterilization cap illuminates to constant lit mode when the temperature in the chamber reaches 104 degrees C. This tells the operator that the chamber is hot. This light begins flashing after the sterilization process is complete, which tells the operator that the sterilization process is complete and the sterilization chamber can be moved to the cooling section. The light goes to constant lit mode again once it has been cooled to a pressure of 41 psi- and the light extinguishes once the temperature reaches 74 degrees Fahrenheit. This tells the operator that the sterilization chamber can be safely removed and unscrewed and the sterilized contents can be safely removed ready for use. The condensed sterile liquid water can then be decanted into a sink or other receptacle for safe disposal according to the particular contaminants it contains.

The chemical indicator that changes color as a result of the sterilization process can be peeled from the spent sterilization water ampoule and pasted into a log book to signify that the handpieces in that load have been sterilized and provide a written record of sterilizations. This requires that the operator chart the serial numbers of the handpieces in the logbook before placing them into the sterilizer for sterilization. Other appropriate chemical indicators can be used.

Collapsible ampoules may be used to facilitate release of water to be converted to saturated steam as the sterilant.

A pressure gradient is established between the insert where the ampoule is placed and the cylinder in which the handpiece resides. Since the water that is to be converted to steam is in one section (the insert) and the cylinder where the handpiece is located has no water to begin with, heating the ampoule with the water in it converts the water to steam and the steam then travels through the handpiece to establish an equilibrium between the two areas thereby sterilizing cleaning and flushing the handpiece.

Following cold spot mapping of the chamber (required for FDA approval as well as prudent sterilization practice) the pressure switch which initiates the sterilization process timing is coordinated with the temperature desired at the cold spot in the sterilization chamber (the head of the handpiece in this case of a dental handpiece) so that the desired conditions can be assured throughout the sterilization chamber. Furthermore, compatibility with the manufacturer's temperature requirements for the instruments to be sterilized can be controlled, thereby not causing damage to the instruments. This temperature and pressure control is illustrated by the graph in FIG. 12. This temperature control can be accomplished with instruments more delicate than dental handpieces--for example proctoscopes which may be able to withstand only lower temperatures. The flexibility of the temperature controller allows us to control the temperature at whatever temperature we desire.

If there are instruments that cannot be subjected to water or steam because, for example, they may rust but can withstand high heat, the present system can be used as a dry heat sterilization system. Instruments can be sterilized using dry heat. The difference is that using dry heat requires a temperature of about 191 degrees C. Attaining this higher temperature can easily be accomplished with the devices described herein as it is easily within the limits of the temperature controllers described.

The loaded sterilization chamber as described herein is so constructed as to suspend the handpieces within the sterilization chamber. In this fashion, the heads of the handpieces, indeed the entirety of the handpieces, encounter only saturated steam as the sterilant. Thus, the entire handpiece is sterilized with saturated steam. Any liquid water within the sterilization chamber is located in the bottom of the sterilization chamber, not in contact with the handpiece. Since saturated water does not have the sterilizing capacity of saturated steam, it needs to be located away from the handpieces.

The sterilization unit described herein is so designed and constructed so that when the loaded sterilization chamber is placed in the heating section, it is oriented at a downward angle from front to back. The heating chamber is purposely oriented in this manner so that any saturated water will run to the downhill side of the chamber away from the instruments to be sterilized. This orientation is also a safety factor for the operator since, once placed into the sterilizer, the sterilization chamber cannot slide out and must be removed by the operator.

The units described herein self-adjust for any thermal load size and for any external barometric pressure (typically a concern at high altitude locations). It self-adjusts because the pressure switch doesn't begin the sterilization process timing count until all the contents within the sterilization chamber have come up to temperature and pressure.

The sterilization of dental handpieces is expected to be an important commercial market for the present devices and processes. The temperature must be controlled to be sufficiently high for a sufficient time to ensure adequate sterilization at all locations on the handpiece, yet not too high so as to damage components of the handpiece. Thus, dental handpieces provide a rather stringent test of the device and its operation. We describe herein by way of illustration and not limitation an embodiment particularly suited for sterilizing dental handpieces.

This embodiment of sterilization device includes a cylindrically-shaped pressure switch, which is normally closed, located mostly within the cap of the sterilization chamber, typically a cap made of black nylon. The pressure switch typically has two wire leads emerging therefrom (top surface) and is threaded with its open port as its bottom surface. This pressure switch is typically about 1.5 inches in height and about 1 inch in diameter. The black nylon cap advantageously contains a printed circuit board including a 10-minute timer programmed into it. The two wire leads from the pressure switch are connected to the printed circuit board. For economy of language, we denote this assembly as the timing logic module. The port of the pressure switch (bottom end, typically threaded) is open to the sterilization chamber cylinder where the contents to be sterilized are located, thereby exposing the pressure switch to the pressure within the sterilization cylinder. The pressure switch is configured to open when the pressure within the cylinder reaches typically 48.5 psi, which concurrently activates the timer within the printed circuit board and is also connected to an indicator light on the printed circuit board.

The threaded port of the pressure switch screws into the internal centerline of a cylindrical metal housing. The external surface of the metal housing is threaded on both ends, one end screws into the sterilization chamber's cylinder while the other end screws into the black nylon cap of the sterilization chamber. When these parts are connected (screwed together) with the sterilization chamber's cylinder, the sterilization chamber is fully formed. This results in the bulk of the pressure switch protruding into the pressure chamber's black nylon cap while the pressure-measuring port of the pressure switch (or the “orifice”) is exposed to the sterilization chamber's cylinder to measure the pressure within the cylinder.

The pressure within the sterilization chamber increases when the chamber is heated. When that pressure reaches a predetermined value (typically 48.5 psi) the pressure switch opens. This opening of the pressure switch activates the printed circuit board through the wire leads connecting the pressure switch to the circuit board, starting the 10-minute timer. This circuit board also conveniently includes an indicator light visible externally through a lens located on the black nylon cap.

The system also includes a temperature switch located within the metal housing next to the pressure switch typically attached to the inner wall of the metal housing but not generally open to the cylinder. This temperature switch is also connected to the printed circuit board such that when the temperature rises to about 50 deg. C, the temperature switch closes and the light is illuminated, thereby informing the operator that the sterilization chamber is hot. On cool down, the temperature switch opens and extinguishes the light when the temperature reaches about room temperature.

Various other modifications and alterations in the structure and method of operation of this invention will be apparent to those skilled in the art without departing from the scope and spirit of the invention. Although the invention has been described in connection with specific embodiments, it should be understood that the invention should not be unduly limited to such specific embodiments.

What is claimed is:

1. A method of sterilizing an implement prior to bringing said implement into contact with a patient comprising:
a) providing a sterilizing device wherein said sterilizing device includes:
a1) a sterilization chamber having the shape of an elongate cartridge suited for containing at least one device to be sterilized; and,
a2) a housing including a heating section and a cooling section thermally insulated from each other, wherein said heating section has a first aperture therein and said cooling section has a second aperture therein, wherein said first and second apertures are suited for receiving said elongate cartridge sequentially into said heating section and said cooling section; and,
a2i) wherein said heating section includes an inner case and at least one inner elongate tubular case in a substantially coaxial configuration, wherein said inner case forms a heat chamber containing said at least one inner elongate tubular case; and,
a2ii) wherein said cooling section includes a cooling tube capable of receiving said elongate cartridge following processing in said heating section; and,
a3) thermally conductive ballast material within said inner case and substantially filling the spaces surrounding said at least one inner elongate tubular case; and,
a4) at least one heater mounted within the space between said inner case and said inner elongate tubular case; and,
a5) a feedback temperature control system including at least one temperature sensor within said inner case or proximate thereto coupled to said at least one heater through said feedback temperature control system wherein said feedback temperature control system includes at least one solid state relay switch and at least one temperature controller; and,
a6) a container within said elongate cartridge wherein said container contains a water sterilizing medium; and,
a7) a pressure switch within said elongate cartridge; and,
b) introducing at least one device to be sterilized into said elongate cartridge; and,
c) introducing a water ampoule into said elongate cartridge and causing said ampoule to be ruptured, releasing said water therein; and,
d) inserting said elongate cartridge into said heating section and heating said water to produce saturated steam at a desired temperature as determined by comparison of measured temperature and pressure with accepted steam tables; and,
e) using said temperature feedback loop to control temperature within a specified range, maintaining said saturated steam in steady state for a desired time interval to ensure adequate sterilization yet not risking damage to heat-sensitive components of said device to be sterilized; and,
f) maintaining exposure of said device to be sterilized to saturated steam in steady state for sufficient time to ensure adequate sterilization; and,
g) transferring said elongate cartridge to said cooling section for sufficient time for the sterilized device to be cooled to a temperature for safe handling after being sterilized in the heating section, optionally including circulating coolant through said cooling section.

2. A method of sterilizing as in claim 1 wherein said feedback temperature control system controls temperature to substantially ±1 degree C.

3. A method of sterilizing as in claim 1, wherein said water is ultrapure water.

4. A method of sterilizing as in claim 1 wherein materials in addition to water are released from said ampoule.

5. A method of sterilizing where said thermally conductive ballast material melts at or near the temperature desired to he delivered Lo said inner elongate tubular case.

6. A method of sterilizing as in claim 1 wherein said elongate cartridge includes a timer that begins a timing cycle when the temperature and pressure reach predetermined values and provides a signal or automatic shut-off following a predetermined time at said desired temperature and pressure.

7. A method of sterilizing as in claim 1 further comprising a fan or blower circulating coolant through said cooling section.

8. A method of sterilizing as in claim 1 further including a pressure relief valve in said elongate cartridge permitting hot steam to be released following completion of the heating process thereby facilitating cooling.
